(19) United States
(12) Patent Application Publication
(10) Pub. No.: US 2011/0039930 A1
(43) Pub. Date: Feb. 17, 2011

(54) FAST-ACTING NAPROXEN COMPOSITION WITH REDUCED GASTROINTESTINAL EFFECTS

(75) Inventors: Michael Novinski, Long Valley, NJ (US); Patrick Osinski, Chatham, NJ (US); Nicholas Hart, Randolph, NJ (US)

Correspondence Address: Jay Lessler;Blank Rome LLP, The Chrysler Building, 405 Lexington Ave., New York, NY, 10174-0208, US

(73) Assignee: Emisphere Technologies, Inc., Cedar Knolls, NJ (US)

(21) Appl. No.: 12/849,527

(22) Filed: Aug. 3, 2010

Related U.S. Application Data
(60) Provisional application No. 61/230,964, filed on Aug. 3, 2009.

(51) Int. Cl. A61K 31/20; A61P 1/00; A61P 29/00; A61K 31/197

(52) U.S. Cl. 514/559; 514/563

(57) ABSTRACT

The present invention relates to pharmaceutical formulations containing Naproxen and a delivery agent.

The present application claims the benefit of U.S. Provisional Application No. 61/230,964, filed Aug. 3, 2009, which is hereby incorporated by reference.

### FIELD OF THE INVENTION

The present invention relates to oral pharmaceutical compositions containing naproxen or a pharmaceutically acceptable salt thereof and a delivery agent.

### BACKGROUND OF THE INVENTION

Naproxen is a propionic acid derivative related to the arylacetic acid group of nonsteroidal anti-inflammatory drugs. It has been widely used to reduce swelling and to treat pain, including dental pain, headache, painful monthly periods, painful joint and muscular problems such as arthritis, tendinitis, bursitis, and gout.

Naproxen is rapidly and completely absorbed from the gastrointestinal tract with an in vivo bioavailability of 95%. The elimination half-life of Naproxen ranges from 12 to 17 hours. Steady-state levels of Naproxen are reached in 4 to 5 days, and the degree of Naproxen accumulation is consistent with this half-life. However, it takes hours to reach peak plasma levels of Naproxen. When given as Naproxen suspension, peak plasma levels of Naproxen are attained in 1 to 4 hours. When given as Naproxen tablets, peak plasma levels of Naproxen are attained in 2 to 4 hours. Moreover, Naproxen causes an increased risk of serious gastrointestinal adverse events including bleeding, ulceration, and perforation of the stomach or intestines, which can be fatal. These events can occur at any time during use and without warning symptoms. Elderly patients are at greater risk for serious gastrointestinal events. There is a need to develop Naproxen formulations which can reach peak plasma levels to provide quick relief and with lower gastrointestinal adverse events.

### SUMMARY OF THE INVENTION

The present invention is an oral pharmaceutical composition (e.g., a tablet or suspension) comprising Naproxen and at least one delivery agent. The pharmaceutical composition provides a faster onset of action of Naproxen to a subject (e.g., a human subject) than a similar composition without the delivery agent, thereby providing faster pain relief and reducing the risk of developing gastric ulcers. Furthermore, the pharmaceutical compositions of the present invention are significantly smaller than fast acting liquid gel capsules containing the same amount of naproxen. The reduced size can improve patient comfort and compliance.

One embodiment of the present invention is an oral pharmaceutical composition comprising Naproxen and at least one delivery agent, such as SNAC, SNAD, 4-CNAB, 5-CNAC, or 4-MOAC. In one preferred embodiment, the oral pharmaceutical composition provides, upon ingestion to a subject (e.g., a healthy human subject), a shortened time period for reaching peak plasma Naproxen levels, compared to a similar composition without the delivery agent.

In one embodiment, the oral pharmaceutical composition includes from about 50 to about 600 mg of Naproxen (calculated on the weight basis of the naproxen base). In other embodiments the oral pharmaceutical composition includes from about 100 to about 400 mg, from about 150 to about 300 mg, or from about 150 to about 250 mg of Naproxen (calculated on the weight basis of the naproxen base). In yet another embodiment, the oral pharmaceutical composition includes about 200 mg of Naproxen (calculated on the weight basis of the naproxen base). For instance, the oral pharmaceutical composition may include about 220 mg of naproxen sodium (equivalent to 200 mg of naproxen base).

The oral pharmaceutical composition may further include another analgesic, such as a 5-HT₁ agonist (e.g., sumatriptan or a pharmaceutically acceptable salt thereof, such as sumatriptan succinate). In one embodiment, the oral pharmaceutical composition includes from about 25 to about 100 mg sumatriptan (e.g., 85 mg) and from about 225 to about 825 mg Naproxen (e.g., 500 mg naproxen sodium).

The oral pharmaceutical composition may further include a proton pump inhibitor, such as omeprazole, esomeprazole, lansoprazole, or any combination thereof. The proton pump inhibitor may be incorporated at a dose sufficient to further reduce the risk of developing gastric ulcers in subjects at risk of developing non-steroidal anti-inflammatory drug (NSAID) associated gastric ulcers.

Another embodiment is a method of providing rapid oral delivery of naproxen to a subject by orally administering an oral pharmaceutical composition of the present invention.

Yet another embodiment is a method of reducing gastrointestinal adverse events caused by oral ingestion of Naproxen in a human subject. The method includes administering an oral pharmaceutical composition of the present invention.

Yet another embodiment is a method of reducing gastrointestinal adverse events caused by oral ingestion of a Naproxen formulation in a human subject. The method includes discontinuing administration of the Naproxen formulation and initiating treatment with an oral pharmaceutical composition of the present invention (e.g., administering an oral pharmaceutical composition of the present invention).

Yet another embodiment is a method of treating pain in a subject in need thereof. The method includes administering an oral pharmaceutical composition of the present invention.

Yet another embodiment is a method of providing relief of the signs and symptoms of rheumatoid arthritis, osteoarthritis, ankylosing spondylitis, juvenile arthritis, tendonitis, bursitis, or acute gout in a subject in need thereof by administering one or more oral pharmaceutical compositions of the present invention. Administration of an effective amount of Naproxen can be achieved with a single composition containing an effective amount of Naproxen or by administration of two or more compositions.

Yet another embodiment is a method of treating pain (e.g., acute pain) or primary dysmenorrheal in a subject in need thereof by administering one or more oral pharmaceutical compositions of the present invention.

Yet another embodiment is a method of treating migraine attacks in a subject in need thereof by administering one or more oral pharmaceutical compositions of the present invention. In one embodiment, the oral pharmaceutical composition further includes another analgesic, such as a 5-HT₁ agonist (e.g., sumatriptan or a pharmaceutically acceptable salt thereof, such as sumatriptan succinate).

In each of the aforementioned methods, oral pharmaceutical compositions of the present invention may be administered once or twice daily to provide a dose of from about 200 to about 600 mg naproxen at each administration. For acute gout, an initial dose of from about 600 to about 1000 mg naproxen (e.g., 750 or 825 mg) followed by from about 200 to about 350 mg naproxen (e.g., 250 or 275 mg) every 8 hours until the attack has subsided.

In one embodiment, one or two oral pharmaceutical compositions of the present invention (e.g., oral pharmaceutical compositions containing 200 mg of Naproxen, for instance 220 mg naproxen sodium) are administered every 8 to 12 hours. In one preferred embodiment, no more than three oral pharmaceutical compositions of the present invention are administered in a 24 hour period.

### DETAILED DESCRIPTION OF THE INVENTION

### Definitions

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean a range of up to 10%, preferably up to 5%.

The terms “alkyl”, “alkenyl”, “alkoxy”, “alkylene”, “alkenylene”, “alkyl(arylene)”, and “aryl(alkylene)” include, but are not limited to, linear and branched alkyl, alkenyl, alkoxy, alkylene, alkenylene, alkyl(arylene), and aryl(alkylene) groups, respectively.

The phrase “pharmaceutically acceptable” refers to compounds or compositions that are physiologically tolerable and do not typically produce an allergic or similar untoward reaction, such as gastric upset, dizziness and the like, when administered to a mammal.

As used herein, the term “peak plasma level” means the maximum concentration reached in the plasma of a mammal, such as a human subject.

The term “bioavailability” refers to the rate and extent to which the active ingredient or active moiety is absorbed from a drug product and becomes systematically available.

The term “polymorph” refers to crystallographically distinct forms of a substance.

The term “hydrate” as used herein includes, but is not limited to, (i) a substance containing water combined in the molecular form and (ii) a crystalline substance containing one or more molecules of water of crystallization or a crystalline material containing free water.

The term “SNAC” as used herein refers to N-(8-[2-hydroxybenzoyl]-amino)caprylic acid and pharmaceutically acceptable salts thereof, including its monosodium and disodium salt. The term “SNAC free acid” refers to N-(8-[2-hydroxybenzoyl]-amino)caprylic acid. Unless otherwise noted, the term “SNAC” refers to all forms of SNAC, including all amorphous and polymorphic forms of SNAC, such as SNAC trihydrate and those described in U.S. Ser. No. 11/568,753 and PCT Application No. PCT/US2005/016126, both of which are hereby incorporated by reference.

The term “SNAD” as used herein refers to N-(10-[2-hydroxybenzoyl]-amino)decanoic acid and pharmaceutically acceptable salts thereof, including its monosodium salt. Unless otherwise noted, the term “SNAD” refers to all forms of SNAD, including all amorphous and polymorphic forms of SNAD.

The term “4-CNAB” as used herein refers to 4-[(4-chloro-2-hydroxy-benzoyl)amino]-butanoic acid (also known as 4-[(2-hydroxy-4-chlorobenzoyl)amino]butanoate) and pharmaceutically acceptable salts thereof, including its sodium salt (e.g., monosodium salt). Unless otherwise noted, the term “4-CNAB” refers to all forms of 4-CNAB, including all amorphous and polymorphic forms of 4-CNAB. The term “sodium 4-CNAB” and “mono-sodium 4-CNAB” refer to monosodium 4-[(2-hydroxy-4-chlorobenzoyl)amino]butanoate, including anhydrous, monohydrate, and isopropanol solvates thereof and amorphous and polymorphic forms thereof such as those described in U.S. Pat. Nos. 7,227,033, 7,208,178, 7,462,368, and 7,420,085.

The term “5-CNAC” as used herein refers to 8-(N-2-hydroxy-5-chlorobenzoyl)amino-caprylic acid and pharmaceutically acceptable salts thereof, including its monosodium and disodium salt. The term “5-CNAC free acid” refers to 8-(N-2-hydroxy-5-chlorobenzoyl)aminocaprylic acid. Unless otherwise noted, the term “5-CNAC” refers to all forms of 5-CNAC, including all amorphous and crystalline forms of 5-CNAC, such as crystalline forms of the disodium salt of 5-CNAC, such as those described in U.S. Patent Publication No. 2008-0269108, which is hereby incorporated by reference.

The term “4-MOAC” as used herein refers to 8-(N-2-hydroxy-4-methoxybenzoyl)-aminocaprylic acid and pharmaceutically acceptable salts thereof, including its monosodium and disodium salt. The term “4-MOAC free acid” refers to 8-(N-2-hydroxy-4-methoxybenzoyl)-aminocaprylic acid. Unless otherwise noted, the term “4-MOAC” refers to all forms of 4-MOAC

The term “delivery agent” refers to any of the delivery agent compounds disclosed or incorporated by reference herein.

The terms “2-OH—Ar” or “2-HO—Ar”, as used in formulas 1 and 2 refers to an aryl group that is substituted with a hydroxy group at the 2 position.

The term “subject” includes mammals and in particular humans.

### Delivery Agent Compounds

Suitable delivery agents include those having the following structure and pharmaceutically acceptable salts thereof:

2-HO—Ar—C(O)—NR⁸—R⁷—COOH Formula (1)

wherein

Ar is phenyl or naphthyl, optionally substituted with OH, halogen, C₁-C₄ alkyl, C₁-C₄ alkenyl, C₁-C₄ alkoxy or C₁-C₄ haloalkoxy;

R⁷ is C₄-C₂₀ alkyl, C₄-C₂₀ alkenyl, phenyl, naphthyl, (C₁-C₁₀ alkyl)phenyl, (C₁-C₁₀ alkenyl)phenyl, (C₁-C₁₀ alkyl)naphthyl, (C₁-C₁₀ alkenyl)naphthyl, phenyl (C₁-C₁₀ alkyl), phenyl (C₁-C₁₀ alkenyl), naphthyl (C₁-C₁₀ alkyl), or naphthyl (C₁-C₁₀ alkenyl);

R⁸ is hydrogen, C₁ to C₄ alkyl, C₂ to C₄ alkenyl, C₁ to C₄ alkoxy, or C₁-C₄ haloalkoxy;

R⁷ is optionally substituted with C₁ to C₄ alkyl, C₂ to C₄ alkenyl, C₁ to C₄ alkoxy, C₁-C₄ haloalkoxy, —OH, —SH, and —CO₂R⁹ or any combination thereof;

R⁹ is hydrogen, C₁ to C₄ alkyl or C₂ to C₄ alkenyl; and

R⁷ is optionally interrupted by oxygen, nitrogen, sulfur or any combination thereof;

with the proviso that the compounds are not substituted with an amino group in the position alpha to the acid group or salts thereof.

According to one embodiment, Ar is substituted with a halogen.

Preferably, R⁷ is C₄-C₂₀ alkyl or phenyl (C₁-C₁₀ alkyl). More preferably, R⁷ is C₅-C₁₀ alkyl or phenyl(C₂ alkyl). Most preferably, R⁷ is C₇-C₉ alkyl or phenyl(C₂ alkyl).

Other suitable delivery agents include those having the following structure and pharmaceutically acceptable salts thereof:

2-OH—Ar—C(O)—NH—R¹—R² Formula (2)

wherein

Ar is phenyl or naphthyl;

Ar is optionally substituted with C₁-C₄ alkyl, C₁-C₄ alkoxy, C₂-C₄ alkenyl, C₂-C₄ alkynyl, aryl, aryloxy, a heterocyclic ring, C₅-C₇ carbocylic ring, halogen, —OH, —SH, CO₂R⁶, —NR⁷R⁸, or —N⁺R⁷R⁸R⁹ Y⁻;

(a) R¹ is C₁-C₁₆ alkylene, C₂-C₁₆ alkenylene, C₂-C₁₆ alkynylene, C₆-C₁₆ arylene, (C₁-C₁₆ alkyl)arylene, or aryl (C₁-C₁₆ alkylene);
R² is —NR³R⁴, or —N⁺R³R⁴R⁵Y⁻;
R³ and R⁴ are independently hydrogen; oxygen; hydroxy; substituted or unsubstituted C₁-C₁₆ alkyl; substituted or unsubstituted C₂-C₁₆ alkenyl; substituted or unsubstituted C₂-C₁₆ alkynyl; substituted or unsubstituted aryl; substituted or unsubstituted alkylcarbonyl; substituted or unsubstituted arylcarbonyl; substituted or unsubstituted alkanesulfinyl; substituted or unsubstituted arylsulfinyl; substituted or unsubstituted alkanesulfonyl; substituted or unsubstituted arylsulfonyl; substituted or unsubstituted alkoxycarbonyl; or substituted or unsubstituted aryloxycarbonyl;
R⁵ is independently hydrogen; substituted or unsubstituted C₁-C₁₆ alkyl; substituted or unsubstituted C₂-C₁₆ alkenyl; substituted or unsubstituted C₂-C₁₆ alkynyl; substituted or unsubstituted aryl; substituted or unsubstituted alkylcarbonyl; substituted or unsubstituted arylcarbonyl; substituted or unsubstituted alkanesulfinyl; substituted or unsubstituted arylsulfinyl; substituted or unsubstituted alkanesulfonyl; substituted or unsubstituted arylsulfonyl; substituted or unsubstituted alkoxycarbonyl; or substituted or unsubstituted aryloxycarbonyl;

(b) R¹, R², and R⁵ are as defined above; and

R³ and R⁴ are combined to form a 5, 6 or 7-membered heterocyclic ring; or 5, 6 or 7-membered heterocyclic ring substituted with a C₁-C₆ alkyl, C₁-C₆ alkoxy, aryl, aryloxy, oxo group or carbocyclic ring; or

(c) R² and R⁵ are as defined above; and

R₁ and R₃ are combined to form a 5, 6 or 7-membered heterocyclic ring; or 5, 6 or 7-membered heterocyclic ring substituted with a C₁-C₆ alkyl, alkoxy, aryl, aryloxy, or oxo group or carbocyclic ring;

R⁴ is hydrogen; oxygen; hydroxy; substituted or unsubstituted C₁-C₁₆ alkyl; substituted or unsubstituted C₂-C₁₆ alkenyl; substituted or unsubstituted C₂-C₁₆ alkynyl; substituted or unsubstituted aryl; substituted or unsubstituted alkylcarbonyl; substituted or unsubstituted arylcarbonyl; substituted or unsubstituted alkanesulfinyl; substituted or unsubstituted arylsulfinyl; substituted or unsubstituted alkanesulfonyl; substituted or unsubstituted arylsulfonyl; substituted or unsubstituted alkoxycarbonyl; or substituted or unsubstituted aryloxycarbonyl;

R⁶ is hydrogen; C₁-C₄ alkyl; C₁-C₄ alkyl substituted halogen or —OH; C₂-C₄ alkenyl; or C₂-C₄ alkenyl substituted halogen or —OH;

R⁷, R⁸, and R⁹ are independently hydrogen; oxygen; C₁-C₄ alkyl; C₁-C₄ alkyl substituted with halogen or —OH; C₂-C₄ alkenyl; or C₂-C₄ alkenyl substituted with halogen or —OH; and

Y is halogen, hydroxide, sulfate, nitrate, phosphate, alkoxy, perchlorate, tetrafluoroborate, or carboxylate. A non-limiting example of a suitable carboxylate is acetate.

The term “substituted” as used herein with respect to the compounds of formula (2) includes, but is not limited to, substitutions with any one or any combination of hydroxyl and halogen.

In one embodiment, Ar is unsubstituted phenyl or phenyl substituted with one or more of C₁-C₄ alkyl, C₁-C₄ alkoxy, or halogen. More preferably, Ar is a phenyl substituted with methoxy, Cl, F or Br, and even more preferably, Ar is a phenyl substituted with Cl.

In another embodiment, R¹ is C₁-C₁₂ alkyl, C₂-C₈ alkyl, C₂-C₆ alkyl, or C₆ alkyl.

In another embodiment, R³ and R⁴ are independently H or C₁-C₂ alkyl; or further R³ and R⁴ are not both H; or further R³ and R⁴ are independently methyl or ethyl; and more preferably R³ and R⁴ are both methyl.

Other suitable delivery agents include those having the following structure and pharmaceutically acceptable salts thereof:

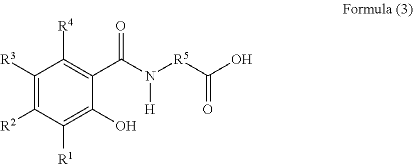

wherein

R¹, R², R³, and R⁴ are independently hydrogen, —OH, —NR⁶R⁷, halogen, C₁-C₄ alkyl, or C₁-C₄ alkoxy;

R⁵ is a substituted or unsubstituted C₂-C₁₆ alkylene, substituted or unsubstituted C₂-C₁₆ alkenylene, substituted or unsubstituted C₁-C₁₂ alkyl(arylene), or substituted or unsubstituted aryl(C₁-C ₁₂ alkylene); and

R⁶ and R⁷ are independently hydrogen, oxygen, or C₁-C₄ alkyl.

The term “substituted” as used with respect to formula (3) includes, but is not limited to, substitution with any one or any combination of the following substituents: halogens, hydroxide, C₁-C₄ alkyl, and C₁-C₄ alkoxy.

Other suitable delivery agents include those having the following structure and pharmaceutically acceptable salts thereof:

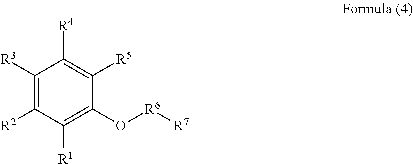

wherein

(a) R¹, R², R³, and R⁴ are independently H, —OH, halogen, C₁-C₄ alkyl, C₁-C₄ alkenyl, C₁-C₄ alkoxy, —C(O)R⁸,—NO₂, —NR⁹R¹⁰, or —N⁺R⁹R¹⁰R¹¹(Y⁻);

R⁸ is hydrogen, —OH, C₁-C₆ alkyl, C₁-C₄ alkyl substituted with halogen or —OH, C₂-C₄ alkenyl unsubstituted or substituted with halogen or —OH, or —NR¹⁴R¹⁵;

R⁹, R¹⁰, and R^{H} are independently hydrogen, oxygen, C₁-C₄ alkyl unsubstituted or substituted with halogen or —OH, C₂-C₄ alkenyl unsubstituted or substituted with halogen or —OH;

Y is halide, hydroxide, sulfate, nitrate, phosphate, alkoxy, perchlorate, tetrafluoroborate, carboxylate, mesylate, fumerate, malonate, succinate, tartrate, acetate, gluconate, or maleate;

R⁵ is H, —OH, —NO₂, halogen, —CF₃, —NR¹⁴R¹⁵, N⁺R¹⁴R¹⁵R¹⁶(Y⁻), amide, C₁-C₁₂ alkoxy, C₁-C₁₂ alkyl, C₂-C₁₂ alkenyl, carbamate, carbonate, urea, or —C(O)R²²; R⁵ is optionally substituted with halogen, —OH, —SH, or —COOH; R⁵ is optionally interrupted by O, N, S, or —C(O)—;

R¹⁴, R¹⁵, and R¹⁶ are independently H or C₁-C₁₀ alkyl;

R²² is H, C₁-C₆ alkyl, —OH, —NR¹⁴R¹⁵;

R⁶ is substituted or unsubstituted C₁-C₁₆ alkylene, C₂-C₁₆ alkenylene, C₂-C₁₆ alkynylene, C₅-C₁₆ arylene, (C₁-C₁₆ alkyl)arylene or aryl(C₁-C₁₆ alkylene); R⁶ is optionally substituted with C₁-C₇ alkyl or C₁-C₇ cycloalkyl;

R⁷ is NR¹⁸R¹⁹ or N⁺R¹⁸R¹⁹R²⁰Y⁻;

R¹⁸ and R¹⁹ are independently hydrogen, oxygen, hydroxy, substituted or unsubstituted C₁-C₁₆ alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl (e.g. substituted or unsubstituted (C₁₋₆ alkyl)carbonyl), substituted or unsubstituted arylcarbonyl, substituted or unsubstituted alkanesulfinyl (e.g. substituted or unsubstituted (C₁₋₆ alkane)sulfinyl), substituted or unsubstituted arylsulfinyl, substituted or unsubstituted alkanesulfonyl (e.g. substituted or unsubstituted (C₁₋₆ alkane)sulfonyl), substituted or unsubstituted arylsulfonyl, substituted or unsubstituted alkoxycarbonyl (e.g. substituted or unsubstituted (C₁₋₆ alkoxy)carbonyl), or substituted or unsubstituted aryloxyccarbonyl, or substituted or unsubstituted C₅-C₇ heterocyclic ring (i.e., 5, 6, or 7-membered heterocyclic ring), wherein the substitutions may be halogen or —OH; and

R²⁰ is independently hydrogen, substituted or unsubstituted C₁-C₁₆ alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl (e.g. substituted or unsubstituted (C₁₋₆ alkyl)carbonyl), substituted or unsubstituted arylcarbonyl, substituted or unsubstituted alkanesulfinyl (e.g. substituted or unsubstituted (C₁₋₆ alkane)sulfinyl), substituted or unsubstituted arylsulfinyl, substituted or unsubstituted alkanesulfonyl (e.g. substituted or unsubstituted (C₁₋₆ alkane)sulfonyl), substituted or unsubstituted arylsulfonyl, substituted or unsubstituted alkoxycarbonyl (e.g. substituted or unsubstituted (C₁₋₆ alkoxy)carbonyl), or substituted or unsubstituted aryloxycarbonyl; or
(b) R¹-R¹⁶ and R²⁰ are as defined above; and
(c) R¹⁸ and R¹⁹ combine to form a 5, 6, or 7-membered heterocyclic ring optionally interrupted with an oxo group and unsubstituted or substituted with C₁-C₆ alkyl, C₁-C₆ alkoxy, aryl, aryloxy, or carbocyclic ring.

According to one embodiment, R⁷ is morpholino, morpholinium salt, or diethanolamino.

According to another embodiment, R⁶ is a C₁-C₁₆ alkylene and R⁷ is morpholino or a morpholinium salt. Preferably, R⁶ is C₄-C₁₂ alkylene, such as an unsubstituted C₄-C₁₂ alkylene. More preferably, R⁶ is C₄-C₁₀, C₄-C₈, or C₆-C₈ alkylene, such as an unsubstituted C₄-C₁₀, C₄-C₈, or C₆-C₈ alkylene. According to one embodiment, one of R¹-R⁵ is hydroxy, for example, R¹ can be hydroxy.

According to yet another embodiment, when R⁶ is a C₁-C₁₀ alkylene, at most one of R² and R⁴ is halogen. According to another embodiment, R⁶ is a C₈-C₁₆, C₉-C₁₆, C₁₀-C₁₆, or C₁₁-C₁₆ alkylene. For instance, R⁶ may be a C₈, C₉, C₁₀, C₁₁, or C₁₂ alkylene (e.g., a normal C₈-C₁₂ alkylene). According to yet another embodiment, at most one of R₁ and R₅ is alkyl.

According to yet another embodiment, R¹ is hydroxy and R², R³, R⁴, and R⁵ are independently hydrogen or halogen.

According to yet another embodiment, R² is hydroxy and R¹, R³, R⁴, and R⁵ are independently hydrogen or halogen.

According to yet another embodiment, R³ is hydroxy and R¹, R², R⁴, and R⁵ are independently hydrogen or halogen.

In a preferred embodiment, halogen is F, Cl or Br, more preferably F or Cl, and even more preferably Cl.

According to yet another embodiment, R⁶ is C₁-C₁₆ alkylene, (C₁-C₁₆ alkyl)arylene or aryl(C₁-C₁₆ alkylene). More preferably R⁶ is C₁-C₁₂ alkylene, more preferably C₃-C₁₀ alkylene, more preferably C₄-C₁₀ or C₄-C₈ alkylene, and more preferably C₆-C₈ alkylene. More preferably, R⁶ is unsubstituted.

According to yet another embodiment, R⁷ is —NR¹⁸R¹⁹ and R¹⁸ and R¹⁹ are independently C₁-C₄ alkyl (e.g., methyl, ethyl, propyl, or butyl) substituted with —OH. In another embodiment, R⁷ is —NR¹⁸R¹⁹ and R¹⁸ and R¹⁹ combine to form a six membered heterocyclic ring substituted with an oxo group.

According to one preferred embodiment, R¹ is hydrogen; R², R³, and R⁴ are independently hydrogen, halogen, —OH, or —OCH₃; R⁵ is hydrogen, —OH, or —C(O)CH₃; R⁶ is C₁-C₁₂ alkylene, and R⁷ is NR¹⁸R¹⁹ wherein R¹⁸ and R¹⁹ combine to form a 5, 6, or 7 membered heterocyclic ring.

According to another preferred embodiment, one of R³, R⁴, and R⁵ is hydroxy and the others are independently halogen or hydrogen; R¹ and R² are independently halogen or hydrogen; R⁶ is C₁-C₁₆ alkylene; and R⁷ is NR¹⁸R¹⁹ wherein R¹⁸ and R¹⁹ combine to form a 5, 6, or 7 membered heterocyclic ring. R⁶ is preferably C₆-C₁₆, C₆-C₁₀, C₈-C₁₆, C₁₀-C₁₆, or C₄-C₈ alkylene, such as unsubstituted C₆-C₁₆, C₆-C₁₀, C₈-C₁₆, C₁₀-C₁₆, or C₄-C₈ alkylene. Preferably, R¹⁸ and R¹⁹ form a morpholino or imidazole.

In another preferred embodiment, R¹ is hydrogen; R², R³, and R⁴ are independently hydrogen, halogen, —OH, or —OCH₃; R⁵ is hydrogen, —OH, or —C(O)CH₃; R⁶ is C₁-C₁₂ alkylene; and R⁷ is N⁺R¹⁸R¹⁹R²⁰ (Y⁻) wherein R¹⁸ and R¹⁹ are hydroxy substituted C₁-C₁₆ alkyl and R²⁰ is hydrogen.

In another preferred embodiment, R¹ is hydrogen; R², R³, and R⁴ are independently hydrogen, halogen, —OH, or —OCH₃; R⁵ is hydrogen, —OH, or —C(O)CH₃; R⁶ is C₁-C₁₂ alkylene; and R⁷ is N⁺R¹⁸R¹⁹R²⁰ (Y⁻) wherein R¹⁸ and R¹⁹ are hydroxyl substituted C₁-C₁₆ alkyl and R²⁰ is hydrogen.

In another preferred embodiment, R¹, R², R⁴, R⁵ are independently halogen or hydrogen; R³ is —OH, or —OCH₃; and R⁷ is N⁺R¹⁸R¹⁹R²⁰ (Y⁻) wherein R¹⁸ and R¹⁹ are hydroxyl substituted C₁-C₁₆ alkyl and R²⁰ is hydrogen.

According to one preferred embodiment, R¹ is hydrogen; R², R³, and R⁴ are independently hydrogen, halogen, —OH, or —OCH₃; R⁵ is hydrogen, —OH, or —C(O)CH₃; R⁶ is C₁-C₆ alkylene or aryl substituted C₁-C₁₂ alkyl; and R⁷ is —NR¹⁸R¹⁹ wherein R¹⁸ and R¹⁹ combine to form a 5, 6, or 7 membered heterocyclic ring or N⁺R¹⁸R¹⁹R²⁰ (Y⁻) wherein R¹⁸ and R¹⁹ are hydroxy substituted C₁-C₁₆ alkyl and R²⁰ is hydrogen.

In another preferred embodiment, the citrate salt of the delivery agent is used.

Other suitable delivery agents include those having the following structure and pharmaceutically acceptable salts thereof:

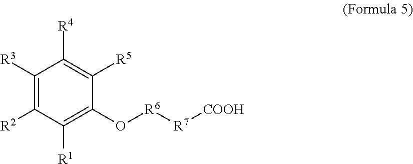

wherein

R¹, R², R³, and R⁴ are independently H, —OH, halogen, C₁-C₄ alkyl, C₂-C₄ alkenyl, C₁-C₄ alkoxy, —C(O)R⁸, —NO₂, —NR⁹R¹⁰, or —N⁺R⁹R¹⁰R¹¹ (R¹³)⁻;

R⁵ is H, —OH, —NO₂, halogen, —CF₃, —NR¹⁴R¹⁵, —N⁺R¹⁴R¹⁵R¹⁶ (R¹³)⁻, amide, C₁-C₁₂ alkoxy, C₁-C₁₂ alkyl, C₂-C₁₂ alkenyl, carbamate, carbonate, urea, or —C(O)R¹⁸;

R⁵ is optionally substituted with halogen, —OH, —SH, or —COOH;

R⁵ is optionally interrupted by O, N, S, or —C(O)—;

R⁶ is a C₁-C₁₂ alkylene, C₂-C₁₂ alkenylene, or arylene;

R⁶ is optionally substituted with a C₁-C₄ alkyl, C₂-C₄ alkenyl, C₁-C₄ alkoxy, —OH, —SH, halogen, —NH₂, or —CO₂R⁸;

R⁶ is optionally interrupted by O or N;

R⁷ is a bond or arylene;

R⁷ is optionally substituted with —OH, halogen, —C(O)CH₃, —NR¹⁰R¹¹, or —N⁺R¹⁰R¹¹R¹² (R¹³)⁻.

R⁸ is H, C₁-C₄ alkyl, C₂-C₄ alkenyl, or —NH₂;

R⁹, R¹⁰, R¹¹, and R¹² independently H or C₁-C₁₀ alkyl;

R¹³ is a halide, hydroxide, sulfate, tetrafluoroborate, or phosphate; and

R¹⁴, R¹⁵ and R¹⁶ are independently H, C₁-C₁₀ alkyl, C₁-C₁₀ alkyl substituted with —COOH, C₂-C₁₂ alkenyl, C₂-C₁₂ alkenyl substituted with —COOH, —C(O)R¹⁷;

R¹⁷ is —OH, C₁-C₁₀ alkyl, or C₂-C₁₂ alkenyl; and

R¹⁸ is H, C₁-C₆ alkyl, —OH, —NR₁₄R¹⁵, or N⁺R¹⁴R¹⁵R¹⁶ (R¹³)⁻.

According one embodiment,

(1) when R¹, R², R³, R⁴, and R⁵ are H, and R⁷ is a bond then R⁶ is not a C₁-C₆, C₉ or C₁₀ alkyl;

(2) when R¹, R², R³, and R⁴ are H, R⁵ is —OH, R⁷ is a bond then R⁶ is not a C₁-C₃ alkyl;

(3) when at least one of R¹, R², R³, and R⁴ is not H, R⁵ is —OH, R⁷ is a bond, then R⁶ is not a C₁-C₄ alkyl;

(4) when R¹, R², and R³ are H, R⁴ is —OCH₃, R⁵ is —C(O)CH₃, and R⁶ is a bond then R⁷ is not a C₃ alkyl; and

(5) when R¹, R², R⁴, and R⁵ are H, R³ is —OH, and R⁷ is a bond then R⁶ is not a methyl.

According one preferred embodiment, R¹ is hydrogen; R², R³, and R⁴ are independently hydrogen, halogen, —OH, or —OCH₃; R⁵ is hydrogen, —OH, or —C(O)CH₃; R⁶ is C₁-C₁₂ alkylene, and R⁷ is a bond or para-phenylene. R⁷ is more preferably a C₇-C₉ alkyl.

According to another preferred embodiment, at least one of R¹, R², R³, and R⁴ is hydrogen, —C(O)CH₃, —OH, Cl, —OCH₃, F, or —NO₂. In one more preferred embodiment, R² is —C(O)CH₃, —OH, —OCH₃, or —Cl. In another more preferred embodiment, R³ is Cl, —OCH₃, F, or —OH. In yet another more preferred embodiment, R⁴ is —OCH₃ or —NO₂.

According to yet another preferred embodiment, R⁵ is —C(O)CH₃, —OH, H, —CH═CHCH₃, —NH₂, —NO₂, —NHC(O)CH₃, —CH═CHCO₂H, —C(O)CH₂CH₃, —C(O)NH₂, —C(O)NHCH₃, —COOH, —C(O)NHCH₂CH₃, —C(O)NHCH(CH₃)₂, —OCH₃, —C(CH₃)₂OH, —C(OH)(CH₃)₂, or —CH(OH)CH₃.

According to yet another preferred embodiment, R⁶ is a linear C₁-C₁₂ alkylene. More preferably, R⁶ is —(CH₂)ₙ—, where n is an integer from 1 to 10.

According to yet another preferred embodiment, R⁴ and R⁵ are not alkyl or halogen.

According to yet another preferred embodiment, R⁷ is para-phenylene or a bond.

According to yet another preferred embodiment, R⁶ is —CH₂ and R⁷ is phenylene and, more preferably para-phenylene. More preferably, at least one of R¹, R², R³, and R⁴ is hydrogen. More preferably, R⁵ is —C(O)CH₃, —OH or —C(CH₃)₂OH.

According to yet another preferred embodiment, R⁷ is a bond, R⁵ is —OH, and R¹, R², R³, and R⁴ are hydrogen. R⁶ is preferably C₄-C₁₂ alkylene and, more preferably, C₄-C₉ alkylene.

According to yet another preferred embodiment, R⁷ is a bond, R⁵ is —OH, and at least one of R¹, R², R³, and R⁴ is not hydrogen. R⁶ is preferably C₁-C₁₂ alkylene, more preferably C₅-C₁₂ alkylene, and most preferably C₅-C₉ alkylene.

According to yet another preferred embodiment, R⁷ is a bond, R⁵ is —C(O)CH₃, and R¹, R², R³, and R⁴ are hydrogen. R⁶ is preferably C₁-C₁₂ alkylene, more preferably C₃-C12 alkylene, and most preferably C₃-C₇ alkylene.

According to yet another preferred embodiment, R⁷ is a bond and R¹, R², R³, R⁴ and R⁵ are hydrogen. Preferably, R⁶ is C₇-C₈ alkylene.

According to yet another preferred embodiment, R⁷ is a bond, R⁵ is hydrogen, and at least one R¹, R², R³, and R⁴ are not hydrogen. R⁶ is preferably C₁-C₁₂ alkylene, more preferably C₄-C₉ alkylene, and most preferably C₇-C₈ alkylene.

According to yet another preferred embodiment, R² is —OH. More preferably, R⁷ is a bond and R⁵ is hydrogen. Preferably, R⁶ is C₁-C₁₂ alkylene, more preferably C₃-C₉ alkylene, and most preferably C₇ alkylene.

According to yet another preferred embodiment, R³ is —OH. More preferably, R⁷ is a bond and R⁵ is hydrogen. R⁶ is preferably C₁-C₁₂ alkylene, more preferably C₃-C₉ alkylene, and most preferably C₇ alkylene.

Other suitable delivery agents include those having the following structure and pharmaceutically acceptable salts thereof:

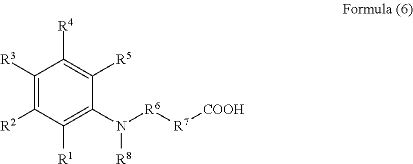

wherein

R¹, R², R³, and R⁴ are independently H, —OH, halogen, —OCH₃, —NR¹⁰R₁₁ or —N⁺R¹⁰R¹¹R¹² (R¹³)⁻;

R⁵ is H, —OH, —NO₂, —NR¹⁴R¹⁵, —N^{+R}¹⁴R¹⁵R¹⁶ (R¹³)₋, amide, C₁-C₁₂ alkoxy, C₁-C₁₂ alkyl, C₂-C₁₂ alkenyl, carbamate, carbonate, urea, or —C(O)R¹⁸;

R⁵ is optionally substituted with —OH, —SH, or —COOH;

R⁵ is optionally interrupted by O, N, S, or —C(O)—;

R⁶ is a C₁-C₁₂ alkylene, C₁-C₁₂ alkenylene, or arylene;

R⁶ is optionally substituted with a C₁-C₄ alkyl, C₂-C₄ alkenyl, C₁-C₄ alkoxy, —OH, —SH, halogen, —NH₂, or —CO₂R⁹;

R⁶ is optionally interrupted by O or N;

R⁷ is a bond or arylene;

R⁷ is optionally substituted with —OH, halogen, —C(O)CH₃, —NR¹⁰R¹¹ or —N⁺R¹⁰R¹¹R¹² (R¹³)⁻.

R⁸ is H or C₁-C₄ alkyl;

R⁹ is H, C₁-C₄ alkyl, or C₂-C₄ alkenyl;

R¹⁰, R¹¹, and R¹² are independently H or C₁-C₁₀ alkyl;

R¹³ is a halide, hydroxide, sulfate, tetrafluoroborate, or phosphate;

R¹⁴, R¹⁵, and R¹⁶ are independently H, C₁-C₁₀ alkyl, C₂-C₁₂ alkenyl, O, or —C(O)R¹⁷;

R¹⁷ is —OH, C₁-C₁₀ alkyl, or C₂-C₁₂ alkenyl; and

R¹⁸ is —OH, C₁-C₆ alkyl, —NR¹⁴R¹⁵, —N⁺R¹⁴R¹⁵R¹⁶ (R¹³)⁻.

According to one embodiment, when R⁵ is OCH₃ then R⁶ is C₁-C₈ or C₁₀-C₁₂ alkyl.

According to a preferred embodiment, R⁵ is not —OCH₃. More preferably, R⁵ is not alkoxy.

According to another preferred embodiment, R¹, R², R³, and R⁴ are hydrogen, R⁵ is —COOH, —C(O)NH₂, —C(O)CH₃, or —NO₂, R⁶ is —(CH₂)_{7—}, and R⁷ is a bond.

According to yet another preferred embodiment, R¹, R², R³, and R⁴ are hydrogen, R⁵ is —C(O)NH₂, R⁶ is —CH₂—, and R⁷ is a para-phenylene.

According to one embodiment, the delivery agents of formula (7) have the formula:

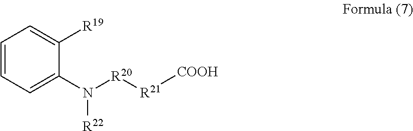

wherein

R¹⁹ is —NO₂ or —C(O)R²³;

R²⁰ is a C₁-C₁₂ alkylene or C₂-C₁₂ alkenylene;

R²¹ is a bond or arylene;

R²² is H or C₁-C₄ alkyl; and

R²³ is —OH, C₁-C₆ alkyl, or —NH₂.

Preferred delivery agents include, but are not limited to, SNAC, SNAD, 5-CNAC, 4-MOAC, 4-CNAB, and pharmaceutically acceptable salts thereof.

According to one preferred embodiment, the delivery agent is SNAC (the free acid or a pharmaceutically acceptable thereof). In one embodiment, the delivery agent is a sodium salt of SNAC. In another embodiment, the delivery agent is the monosodium salt of SNAC and can be, for example, any of the solid state forms of monosodium SNAC disclosed in U.S. application Ser. No. 11,568,753 and PCT Application No. PCT/US2005/016126, both of which are hereby incorporated by reference. In yet another embodiment, the delivery agent is the disodium salt of SNAC.

According to another preferred embodiment, the delivery agent is SNAD (the free acid or a pharmaceutically acceptable thereof). In one embodiment, the delivery agent is a sodium salt of SNAD. In another embodiment, the delivery agent is the disodium salt of SNAD.

According to yet another preferred embodiment, the delivery agent is 4-CNAB (the free acid or a pharmaceutically acceptable thereof). In one embodiment, the delivery agent is a sodium salt of 4-CNAB. The sodium 4-CNAB can be any of the amorphous and polymorphic forms described in International Publication No. WO 03/057650, which is hereby incorporated by reference.

Other suitable delivery agents of the present invention are described in U. S. Pat. Nos. 6,699,467, 6,663,898, 6,693,208, 6,693,073, 6,693,898, 6,663,887, 6,646,162, 6,642,411, 6,627,228, 6,623,731, 6,610,329, 6,558,706, 6,525,020, 6,461,643, 6,461,545, 6,440,929, 6,428,780, 6,413,550, 6,399,798, 6,395,774, 6,391,303, 6,384,278, 6,375,983, 6,358,504, 6,346,242, 6,344,213, 6,331,318, 6,313,088, 6,245,359, 6,242,495, 6,221,367, 6,180,140, 6,100,298, 6,100,285, 6,099,856, 6,090,958, 6,084,112, 6,071,510, 6,060,513, 6,051,561, 6,051,258, 6,001,347, 5,990,166, 5,989,539, 5,976,569, 5,972,387, 5,965,121, 5,962,710, 5,958,451, 5,955,503, 5,939,381, 5,935,601, 5,879,681, 5,876,710, 5,866,536, 5,863,944, 5,840,340, 5,824,345, 5,820,881, 5,811,127, 5,804,688, 5,792,451, 5,776,888, 5,773,647, 5,766,633, 5,750,147, 5,714,167, 5,709,861, 5,693,338, 5,667,806, 5,650,386, 5,643,957, 5,629,020, 5,601,846, 5,578,323, 5,541,155, 5,540,939, 5,451,410, 5,447,728, 5,443,841, and 5,401,516. Delivery agents of the present invention are also described in U.S. Published Application Nos. 20040110839, 20040106825, 20040068013, 20040062773, 20040022856, 20030235612, 20030232085, 20030225300, 20030198658, 20030133953, 20030078302, 20030072740, 20030045579, 20030012817, 20030008900, 20020155993, 20020127202, 20020120009, 20020119910, 20020102286, 20020065255, 20020052422, 20020040061, 20020028250, 20020013497, 20020001591, 20010039258, and 20010003001. Delivery agents of the present invention are also described in International Publication Nos. WO 2004/4104018, WO 2004080401, WO 2004062587, WO 2003/057650, WO 2003/057170, WO 2003/045331, WO 2003/045306, WO 2003/026582, WO 2002/100338, WO 2002/070438, WO 2002/069937, WO 02/20466, WO 02/19969, WO 021/6309, WO 02/15959, WO 02/02509, WO 01/92206, WO 01/70219, WO 01/51454, WO 01/44199, WO 01/34114, WO 01/32596, WO 01/32130, WO 00/07979, WO 00/06534, WO 00/06184, WO 00/59863, WO 00/59480, WO 00/50386, WO 00/48589, WO 00/47188, WO 00/46182, WO 00/40203, WO 99/16427, WO 98/50341, WO 98/49135, WO 98/34632, WO 98/25589, WO 98/21951, WO 97/47288, WO 97/31938, WO 97/10197, WO 96/40076, WO 96/40070, WO 96/39835, WO 96/33699, WO 96/30036, WO 96/21464, WO 96/12475, and WO 9612474. Each of the above listed U.S. patents and U.S. and International published applications are herein incorporated by reference.

The delivery agent compounds depicted as carboxylic acids may be in the form of the carboxylic acid or salts thereof. Suitable salts include, but are not limited to, organic and inorganic salts, for example alkali-metal salts, such as sodium (e.g., monosodium and disodium salts), potassium and lithium; alkaline-earth metal salts, such as magnesium, calcium or barium; ammonium salts; basic amino acids, such as lysine or arginine; and organic amines, such as dimethylamine or pyridine. Preferably, the salts are sodium salts. The salts may be mono-or multi-valent salts, such as monosodium salts and di-sodium salts. The salts may also be solvates, including ethanol solvates, and hydrates.

The delivery agent compounds depicted as amines may be in the form of the free amine or salts thereof. Suitable salts include, but are not limited to, organic and inorganic salts, for example sodium salts, sulfate salts, hydrochloride salts, phosphate salts, fluoride salts, carbonate salts, tartrate salts, oxalates, oxides, formates, acetate or citrate.

Salts of the delivery agent compounds of the present invention may be prepared by methods known in the art. For example, sodium salts may be prepared by dissolving the delivery agent compound in ethanol and adding aqueous sodium hydroxide.

Where the delivery agent has an amine moiety and a carboxylic acid moiety, poly amino acids and peptides comprising one or more of these compounds may be used. An amino acid is any carboxylic acid having at least one free amine group and includes naturally occurring and synthetic amino acids. Poly amino acids are either peptides (which are two or more amino acids joined by a peptide bond) or are two or more amino acids linked by a bond formed by other groups which can be linked by, e.g., an ester or an anhydride linkage. Peptides can vary in length from dipeptides with two amino acids to polypeptides with several hundred amino acids. One or more of the amino acids or peptide units may be acylated or sulfonated.

The delivery agent may contain a polymer conjugated to it such as described in International Publication No. WO 03/045306. For example, the delivery agent and polymer may be conjugated by a linkage group selected from the group consisting of —NHC(O)NH—, —C(O)NH—, —NHC(O), —OOC—, —COO—, NHC(O)O—, —OC(O)NH—, CH2NH—NHCH2—, —CH₂NHC(O)O—, —OC(O)NHCH₂—, —CH₂NHCOCH₂O—, —OCH₂C(O)NHCH₂—, NHC(O)CH₂O—, —OCH₂C(O)NH—, —NH—, —OP, and carbon-carbon bond, with the proviso that the polymeric delivery agent is not a polypeptide or polyamino acid. The polymer may be any polymer including, but not limited to, alternating copolymers, block copolymers and random copolymers, which are safe for use in mammals.

Preferred polymers include, but are not limited to, polyethylene; polyacrylates; polymethacrylates; poly (oxyethylene); poly (propylene); polypropylene glycol; polyethylene glycol (PEG); and derivatives thereof and combinations thereof. The molecular weight of the polymer typically ranges from about 100 to about 200,000 daltons. The molecular weight of the polymer preferably ranges from about 200 to about 10,000 daltons. In one embodiment, the molecular weight of the polymer ranges from about 200 to about 600 daltons and more preferably ranges from about 300 to about 550 daltons.

The compounds described herein may be derived from amino acids and can be readily prepared from amino acids by methods within the skill of those in the art, such as those described in International Publication Nos. W096/30036, W097/36480, WO 00/06534, WO 00/46812, WO 00/50386, WO 00/59863, WO 01/32596, and WO 00/07979 and U.S. Pat. Nos. 5,643,957, 5,650,386, and 5,866,536, all of which are incorporated by reference. For example, the compounds may be prepared by reacting the single amino acid with the appropriate acylating or amine-modifying agent, which reacts with a free amino moiety present in the amino acid to form amides. Protecting groups may be used to avoid unwanted side reactions as would be known to those skilled in the art. With regard to protecting groups, reference is made to T. W. Greene, Protecting Groups in Organic Synthesis, Wiley, New York (1981), the disclosure of which is hereby incorporated herein by reference.

The delivery agent compound may be purified by recrystallization or by fractionation on one or more solid chromatographic supports, alone or linked in tandem. Suitable recrystallization solvent systems include, but are not limited to, acetonitrile, methanol, ethanol, ethyl acetate, heptane, water, tetrahydrofuran, and combinations thereof. Fractionation may be performed on a suitable chromatographic support such as alumina, using methanol/n-propanol mixtures as the mobile phase; reverse phase chromatography using trifluoroacetic acid/acetonitrile mixtures as the mobile phase; and ion exchange chromatography using water or an appropriate buffer as the mobile phase. When anion exchange chromatography is performed, preferably a 0-500 mM sodium chloride gradient is employed.

### Naproxen

Naproxen means 2-(6-methoxynaphthalen-2-yl)propanoic acid and pharmaceutically acceptable salts thereof, including its sodium salt. The term “Naproxen free acid” refers to 2-(6-methoxynaphthalen-2-yl)propanoic acid. Unless otherwise noted, the term “Naproxen” refers to all forms of Napraxen, including all amorphous and crystalline forms of Naproxen.

Solid pharmaceutical compositions may be in the form of tablets, capsules (including hard and soft gelatin capsules), and particles, such as powders and sachets. An oral tablet is a preferred dosage form of the present invention. Solid dosage forms may be prepared by mixing the solid form of the delivery agent with the solid form of the incretin hormone. Alternately, a solid may be obtained from a solution of delivery agent and incretin hormone by methods known in the art, such as freeze-drying (lyophilization), precipitation, crystallization and solid dispersion.

The pharmaceutical compositions can include anyone or combination of excipients, diluents, disintegrants, lubricants, fillers, plasticizers, colorants, flavorants, taste-masking agents, sugars, sweeteners and salts.

In one embodiment, the weight ratio of delivery agent to Naproxen is from about 1:8 to about 2:1. In another embodiment, the weight ratio of delivery agent to Naproxen is from about 1:6 to about 1.5:1, from about 1:4 to about 1:1.25, or from about 1:4.5 to about 1:1.

In one embodiment, the delivery agent is SNAC, SNAD, 5-CNAC, 4-MOAC, or 4-CNAB and the weight ratio of delivery agent and Naproxen is between 1:5 and 20:1, preferably between 1:3 and 15:1, more preferably between 1:1 and 10:1, and the most preferably between 2:1 and 5:1.

In yet another embodiment, the delivery agent is SNAC, SNAD, 5-CNAC, 4-MOAC, or 4-CNAB, the Naproxen is naproxen sodium, and the weight ratio of SNAC, SNAD or 4-CNAB to naproxen sodium is about 1:4.4, about 1:2.2, or about 1:1.1.

In yet another embodiment, the delivery agent is the monosodium salt of SNAC, the Naproxen is naproxen sodium, and the weight ratio of the monosodium salt of SNAC to naproxen sodium is about 1:4.4, about 1:2.2, or about 1:1.1.

In another embodiment, the pharmaceutical composition contains from about 25 mg to about 500 mg of Naproxen and from about 50 mg to about 600 mg of delivery agent. Preferably, the pharmaceutical composition, upon oral ingestion to a human, provides peak plasma levels of Naproxen in 60 minutes or less, more preferably in 45 minutes or less, and the most preferably in 30 minutes or less.

In yet another embodiment, the pharmaceutical composition includes from about 25 mg to about 500 mg of Naproxen and from about 50 mg to about 600 mg of the monosodium salt of SNAC. For example, the pharmaceutical composition may include from about 150 to about 300 mg of Naproxen (preferably naproxen sodium) (calculated based on weight of naproxen base) and from about 50 to about 200 mg of SNAC (preferably the monosodium salt of SNAC).

The pharmaceutical compositions are useful for treating pains with reduced gastrointestinal adverse events caused by oral ingestion of Naproxen in a human subjects. Preferably the gastrointestinal adverse events are reduced by more than 20% as compared with administration of Naproxen alone, more preferably by more than 40% and the most preferably by more than 60%.

### Example 1

Naproxen tablets are made and each tablet contains 110 mg of Naproxen and 250 mg of SNAC. These tablets reach peak Naproxen levels at much shorter time as compared with Naproxen tablets that do not contain SNAC.

All publications, patents, and patent applications cited herein are hereby incorporated by reference.

1. An oral pharmaceutical composition comprising (a) Naproxen and (b) at least one delivery agent selected from the following compounds, and pharmaceutically acceptable salts thereof:
2-HO—Ar—C(O)—NR⁸—R⁷—COOH Formula (1)
wherein
Ar is phenyl or naphthyl, optionally substituted with OH, halogen, C₁-C₄ alkyl, C₁-C₄ alkenyl, C₁-C₄ alkoxy or C₁-C₄ haloalkoxy;
R⁷ is C₄-C₂₀ alkyl, C₄-C₂₀ alkenyl, phenyl, naphthyl, (C₁-C₁₀ alkyl)phenyl, (C₁-C₁₀ alkenyl)phenyl, (C₁-C₁₀ alkyl)naphthyl, (C₁-C₁₀ alkenyl)naphthyl, phenyl (C₁-C₁₀ alkyl), phenyl (C₁-C₁₀ alkenyl), naphthyl (C₁-C₁₀ alkyl), or naphthyl (C₁-C₁₀ alkenyl);
R⁸ is hydrogen, C₁ to C₄ alkyl, C₂ to C₄ alkenyl, C₁ to C₄ alkoxy, or C₁-C₄ haloalkoxy;
R⁷ is optionally substituted with C₁ to C₄ alkyl, C₂ to C₄ alkenyl, C₁ to C₄ alkoxy, C₁-C₄ haloalkoxy, —OH, —SH, and —CO₂R⁹ or any combination thereof;
R⁹ is hydrogen, C₁ to C₄ alkyl or C₂ to C₄ alkenyl; and
R⁷ is optionally interrupted by oxygen, nitrogen, sulfur or any combination thereof;
with the proviso that the compounds are not substituted with an amino group in the position alpha to the acid group or salts thereof;
2-OH—Ar—C(O)—NH—R¹-R² Formula (2)
wherein
Ar is phenyl or naphthyl;
Ar is optionally substituted with C₁-C₄ alkyl, C₁-C₄ alkoxy, C₂-C₄ alkenyl, C₂-C₄ alkynyl, aryl, aryloxy, a heterocyclic ring, C₅-C₇ carbocylic ring, halogen, —OH, —SH, CO₂R⁶, —NR⁷R⁸, or —N⁺R⁷R⁸R⁹Y⁻;
(a) R¹ is C₁-C₁₆ alkylene, C₂-C₁₆ alkenylene, C₂-C₁₆ alkynylene, C₆-C₁₆ arylene, (C₁-C₁₆ alkyl)arylene, or aryl (C₁-C₁₆ alkylene);
R² is —NR³R⁴, or —N⁺R³R⁴R⁵Y⁻;
R³ and R⁴ are independently hydrogen; oxygen; hydroxy; substituted or unsubstituted C₁-C₁₆ alkyl; substituted or unsubstituted C₂-C₁₆ alkenyl; substituted or unsubstituted C₂-C₁₆ alkynyl; substituted or unsubstituted aryl; substituted or unsubstituted alkylcarbonyl; substituted or unsubstituted arylcarbonyl; substituted or unsubstituted alkanesulfinyl; substituted or unsubstituted arylsulfinyl; substituted or unsubstituted alkanesulfonyl; substituted or unsubstituted arylsulfonyl; substituted or unsubstituted alkoxycarbonyl; substituted or unsubstituted aryloxycarbonyl;
R⁵ is independently hydrogen; substituted or unsubstituted C₁-C₁₆ alkyl; substituted or unsubstituted C₂-C₁₆ alkenyl; substituted or unsubstituted C₂-C₁₆ alkynyl;
substituted or unsubstituted aryl; substituted or unsubstituted alkylcarbonyl; substituted or unsubstituted arylcarbonyl; substituted or unsubstituted alkanesulfinyl; substituted or unsubstituted arylsulfinyl; substituted or unsubstituted alkanesulfonyl; substituted or unsubstituted arylsulfonyl; substituted or unsubstituted alkoxycarbonyl; substituted or unsubstituted aryloxycarbonyl;
(b) R¹, R², and R⁵ are as defined above; and
R³ and R⁴ are combined to form a 5, 6 or 7-membered heterocyclic ring; or 5, 6 or 7-membered heterocyclic ring substituted with a C₁-C₆ alkyl, C₁-C₆ alkoxy, aryl, aryloxy, oxo group or carbocyclic ring; or
(c) R² and R⁵ are as defined above; and
R₁ and R₃ are combined to form a 5, 6 or 7-membered heterocyclic ring; or 5, 6 or 7-membered heterocyclic ring substituted with a C₁-C₆ alkyl, alkoxy, aryl, aryloxy, or oxo group or carbocyclic ring;
R⁴ is hydrogen; oxygen; hydroxy; substituted or unsubstituted C₁-C₁₆ alkyl; substituted or unsubstituted C₂-C₁₆ alkenyl; substituted or unsubstituted C₂-C₁₆ alkynyl; substituted or unsubstituted aryl; substituted or unsubstituted alkylcarbonyl; substituted or unsubstituted arylcarbonyl; substituted or unsubstituted alkanesulfinyl; substituted or unsubstituted arylsulfinyl; substituted or unsubstituted alkanesulfonyl; substituted or unsubstituted arylsulfonyl; substituted or unsubstituted alkoxycarbonyl; substituted or unsubstituted aryloxycarbonyl;
R⁶ is hydrogen; C₁-C₄ alkyl; C₁-C₄ alkyl substituted halogen or —OH; C₂-C₄ alkenyl; or C₂-C₄ alkenyl substituted halogen or —OH;
R⁷, R⁸, and R⁹ are independently hydrogen; oxygen; C₁-C₄ alkyl; C₁-C₄ alkyl substituted with halogen or —OH; C₂-C₄ alkenyl; or C₂-C₄ alkenyl substituted with halogen or —OH; and
Y is halogen, hydroxide, sulfate, nitrate, phosphate, alkoxy, perchlorate, tetrafluoroborate, or caboxylate; 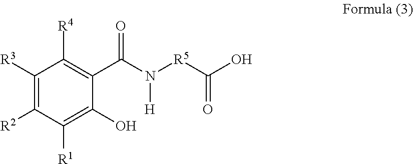
wherein
R¹, R², R³, and R⁴ are independently hydrogen, —OH, —NR⁶R⁷, halogen, C₁-C₄ alkyl, or C₁-C₄ alkoxy;
R⁵ is a substituted or unsubstituted C₂-C₁₆ alkylene, substituted or unsubstituted C₂-C₁₆ alkenylene, substituted or unsubstituted C₁-C₁₂ alkyl(arylene), or substituted or unsubstituted aryl(C₁-C₁₂ alkylene); and
R⁶ and R⁷ are independently hydrogen, oxygen, or C₁-C₄ alkyl; 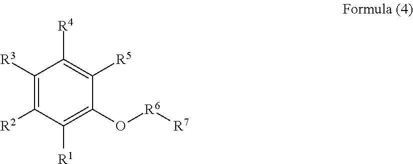
wherein
(a) R¹, R², R³, and R⁴ are independently H, —OH, halogen, C₁-C₄ alkyl, C₁-C₄ alkenyl, C₁-C₄ alkoxy, —C(O)R⁸, —NO₂, —NR⁹R¹⁰, or —N⁺R⁹R¹⁰R¹¹(Y^{—});
R⁸ is hydrogen, —OH, C₁-C₆ alkyl, C₁-C₄ alkyl substituted with halogen or —OH, C₂-C₄ alkenyl unsubstituted or substituted with halogen or —OH, or —NR¹⁴R¹⁵;
R⁹, R¹⁰, and R¹¹ are independently hydrogen, oxygen, C₁-C₄ alkyl unsubstituted or substituted with halogen or —OH, C₂-C₄ alkenyl unsubstituted or substituted with halogen or —OH;
Y is halide, hydroxide, sulfate, nitrate, phosphate, alkoxy, perchlorate, tetrafluoroborate, carboxylate, mesylate, fumerate, malonate, succinate, tartrate, acetate, gluconate, or maleate;
R⁵ is H, —OH, —NO₂, halogen, —CF₃, —NR¹⁴R¹⁵, —N⁺R¹⁴R¹⁵R¹⁶(Y⁻), amide, C₁-C₁₂ alkoxy, C₁-C₁₂ alkyl, C₂-C₁₂ alkenyl, carbamate, carbonate, urea, or —C(O)R²²; R⁵ is optionally substituted with halogen, —OH, —SH, or —COOH; R⁵ is optionally interrupted by O, N, S, or —C(O)—;
R¹⁴, R¹⁵, and R¹⁶ are independently H or C₁-C₁₀ alkyl;
R²² is H, C₁-C₆ alkyl, —OH, —NR¹⁴R¹⁵;
R⁶ is substituted or unsubstituted C₁-C₁₆ alkylene, C₂-C₁₆ alkenylene, C₂-C₁₆ alkynylene, C₅-C₁₆ arylene, (C₁-C₁₆ alkyl)arylene or aryl(C₁-C₁₆ alkylene); R⁶ is optionally substituted with C₁-C₇ alkyl or C₁-C₇ cycloalkyl;
R⁷ is —NR¹⁸R¹⁹ or N⁺R¹⁸R¹⁹R²⁰Y⁻;
R¹⁸ and R¹⁹ are independently hydrogen, oxygen, hydroxy, substituted or unsubstituted C₁-C₁₆ alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl (e.g. substituted or unsubstituted (C₁₋₆ alkyl)carbonyl), substituted or unsubstituted arylcarbonyl, substituted or unsubstituted alkanesulfinyl (e.g. substituted or unsubstituted (C₁₋₆ alkane)sulfinyl), substituted or unsubstituted arylsulfinyl, substituted or unsubstituted alkanesulfonyl (e.g. substituted or unsubstituted (C₁₋₆ alkane)sulfonyl), substituted or unsubstituted arylsulfonyl, substituted or unsubstituted alkoxycarbonyl (e.g. substituted or unsubstituted (C₁₋₆ alkoxy)carbonyl), or substituted or unsubstituted aryloxyccarbonyl, or substituted or unsubstituted C₅-C₇ heterocyclic ring (i.e., 5, 6, or 7-membered heterocyclic ring), wherein the substitutions may be halogen or —OH; and
R²⁰ is independently hydrogen, substituted or unsubstituted C₁-C₁₆ alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl (e.g. substituted or unsubstituted (C₁₋₆ alkyl)carbonyl), substituted or unsubstituted arylcarbonyl, substituted or unsubstituted alkanesulfinyl (e.g. substituted or unsubstituted (C₁₋₆ alkane)sulfinyl), substituted or unsubstituted arylsulfinyl, substituted or unsubstituted alkanesulfonyl (e.g. substituted or unsubstituted (C₁₋₆ alkane)sulfonyl), substituted or unsubstituted arylsulfonyl, substituted or unsubstituted alkoxycarbonyl (e.g. substituted or unsubstituted (C₁₋₆ alkoxy)carbonyl), or substituted or unsubstituted aryloxycarbonyl; or
(b) R¹-R¹⁶ and R²⁰ are as defined above; and
R¹⁸ and R¹⁹ combine to form a 5, 6, or 7-membered heterocyclic ring optionally interrupted with an oxo group and unsubstituted or substituted with C₁-C₆ alkyl, C₁-C₆ alkoxy, aryl, aryloxy, or carbocyclic ring; 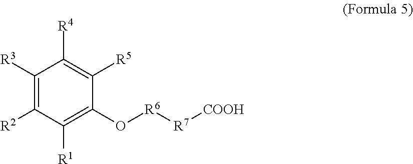
wherein
R¹, R², R³, and R⁴ are independently H, —OH, halogen, C₁-C₄ alkyl, C₂-C₄ alkenyl, C₁-C₄ alkoxy, —C(O)R⁸, —NO₂, —NR⁹R¹⁰, or —N⁺R⁹R¹⁰R¹¹ (R¹²)⁻;
R⁵ is H, —OH, —NO₂, halogen, —CF₃, —NR¹⁴R¹⁵, —N⁺R¹⁴R¹⁵R¹⁶ (R¹³)⁻, amide, C₁-C₁₂ alkoxy, C₁-C₁₂ alkyl, C₂-C₁₂ alkenyl, carbamate, carbonate, urea, or —C(O)R¹⁸;
R⁵ is optionally substituted with halogen, —OH, —SH, or —COOH;
R⁵ is optionally interrupted by O, N, S, or —C(O)—;
R⁶ is a C₁-C₁₂ alkylene, C₂-C₁₂ alkenylene, or arylene;
R⁶ is optionally substituted with a C₁-C₄ alkyl, C₂-C₄ alkenyl, C₁-C₄ alkoxy, —OH, —SH, halogen, —NH₂, or —CO₂R⁸;
R⁶ is optionally interrupted by O or N;
R⁷ is a bond or arylene;
R⁷ is optionally substituted with —OH, halogen, —C(O)CH₃, —NR¹⁰R¹¹, or —N⁺R¹⁰R¹¹R¹² (R¹³)⁻;
R⁸ is H, C₁-C₄ alkyl, C₂-C₄ alkenyl, or —NH₂;
R⁹, R¹⁰, R¹¹, and R¹² independently H or C₁-C₁₀ alkyl;
R¹³ is a halide, hydroxide, sulfate, tetrafluoroborate, or phosphate; and
R¹⁴, R¹⁵ and R¹⁶ independently H, C₁-C₁₀ alkyl, C₁-C₁₀ alkyl substituted with COOH, C₂-C₁₂ alkenyl, C₂-C₁₂ alkenyl substituted with —COOH, —C(O)R¹⁷;
R¹⁷ is —OH, C₁-C₁₀ alkyl, or C₂-C₁₂ alkenyl; and
R¹⁸ is H, C₁-C₆ alkyl, —OH, —NR¹⁴R¹⁵, or N⁺R¹⁴R¹⁵R¹⁶ (R¹³); 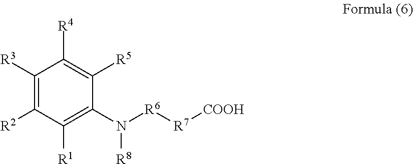
wherein
R¹, R², R³ , and R⁴ are independently H, —OH, halogen, —OCH₃, —NR¹⁰R¹¹ or —N⁺R¹⁰R¹¹R¹² (R¹³)⁻;
R⁵ is H, —OH, —NO₂, —NR¹⁴R¹⁵, —N⁺R¹⁴R¹⁵R¹⁶ (R¹³)⁻; amide, C₁-C₁₂ alkoxy, C₁-C₁₂ alkyl, C₂-C₁₂ alkenyl, carbamate, carbonate, urea, or —C(O)R¹⁸;
R⁵ is optionally substituted with —OH, —SH, or —COOH;
R⁵ is optionally interrupted by O, N, S, or —C(O)—;
R⁶ is a C₁-C₁₂ alkylene, C₁-C₁₂ alkenylene, or arylene;
R⁶ is optionally substituted with a C₁-C₄ alkyl, C₂-C₄ alkenyl, C₁-C₄ alkoxy, —OH, —SH, halogen, —NH₂, or —CO₂R⁹;
R⁶ is optionally interrupted by O or N;
R⁷ is a bond or arylene;
R⁷ is optionally substituted with —OH, halogen, —C(O)CH₃, —NR¹⁰R₁₁ or —N⁺R¹⁰R¹¹R¹² (R¹³)⁻;
R⁸ is H or C₁-C₄ alkyl;
R⁹ is H, C₁-C₄ alkyl, or C₂-C₄ alkenyl;
R¹⁰, R¹¹, and R¹² are independently H or C₁-C₁₀ alkyl;
R¹³ is a halide, hydroxide, sulfate, tetrafluoroborate, or phosphate;
R¹⁴, R¹⁵, and R¹⁶ are independently H, C₁-C₁₀ alkyl, C₂-C₁₂ alkenyl, O, or —C(O)R^{1y};
R¹⁷ is —OH, C₁-C₁₀ alkyl, or C₂-C₁₂ alkenyl; and
R¹⁸ is —OH, C₁-C₆ alkyl, —NR¹⁴R¹⁵, —N⁺R¹⁴R¹⁵R¹⁶ (R¹³)⁻; 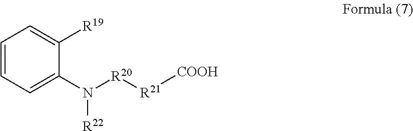
wherein
R¹⁹ is —NO₂ or —C(O)R²³;
R²⁰ is a C₁-C₁₂ alkylene or C₂-C₁₂ alkenylene;
R²¹ is a bond or arylene;
R²² is H or C₁-C₄ alkyl; and
R²³ is —OH, C₁-C₆ alkyl, or —NH₂.

2. The pharmaceutical composition of claim 1, wherein said delivery agent is SNAC.

3. The pharmaceutical composition of claim 2, wherein the weight ratio of said SNAC and said Naproxen is between 1:5 and 20:1.

4. The pharmaceutical composition of claim 2, wherein the weight ratio of said SNAC and said Naproxen is between 1:3 and 15:1.

5. The pharmaceutical composition of claim 2, wherein the weight ratio of said SNAC and said Naproxen is between 1:1 and 10:1.

6. The pharmaceutical composition of claim 2, wherein the weight ratio of said SNAC and said Naproxen is between 2:1 and 5:1.

7. The pharmaceutical composition of claim 1, wherein said delivery agent is SNAD.

8. The pharmaceutical composition of claim 1, wherein said delivery agent is 4-CNAB.

9. The pharmaceutical composition of claim 1, wherein said pharmaceutical composition contains from about 25 mg to about 500 mg of Naproxen.

10. The pharmaceutical composition of claim 1, wherein said pharmaceutical composition contains from about 50 mg to about 600 mg of delivery agent.

11. The pharmaceutical composition of claim 1, wherein said pharmaceutical composition, upon oral ingestion to a human, provides peak plasma levels of Naproxen in 60 minutes or less.

12. The pharmaceutical composition of claim 1, wherein said pharmaceutical composition, upon oral ingestion to a human, provides peak plasma Naproxen in 45 minutes or less.

13. The pharmaceutical composition of claim 1, wherein said pharmaceutical composition, upon oral ingestion to a human, provides peak plasma Naproxen in 30 minutes or less.

14. A method of reducing gastrointestinal adverse events caused by oral ingestion of Naproxen in a human subject comprising the step of administering the pharmaceutical composition of claim 1 to said human subject.

15. The method of claim 17, wherein said gastrointestinal adverse events are reduced by more than 20%.

16. The method of claim 17, wherein said gastrointestinal adverse events are reduced by more than 40%.

17. The method of claim 17, wherein said gastrointestinal adverse events 15 are reduced by more than 60%.

18. A method of treating pain in a subject in need thereof, comprising administering the oral pharmaceutical composition of any of claim 1.

19. A method of treating pain in a subject in need thereof comprising administering an effective amount of an oral pharmaceutical composition comprising from about 150 to about 300 mg of naproxen or a pharmaceutically acceptable salt thereof (calculated on the weight basis of naproxen base) and from about 50 to about 200 mg of N-(8-[2-hydroxybenzoyl]amino)caprylic acid or a pharmaceutically acceptable salt thereof.

20. The method of claim 19, wherein the oral pharmaceutical composition comprises about 220 mg naproxen sodium and from about 50 to about 200 mg of a monosodium salt of N-(8-[2-hydroxybenzoyl]amino)caprylic acid, and the oral pharmaceutical composition is a tablet.
